(19) United States
(12) Patent Application Publication
(10) Pub. No.: US 2009/0018524 A1
(43) Pub. Date: Jan. 15, 2009

(54) EXPANDABLE DELIVERY DEVICE

(75) Inventors: E. Skott GREENHALGH, Lower Gwynedd, PA (US); John-Paul ROMANO, Chalfont, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 12/139,367

(22) Filed: Jun. 13, 2008

Related U.S. Application Data
(63) Continuation of application No. PCT/US2006/062337, filed on Dec. 19, 2006.
(60) Provisional application No. 60/751,882, filed on Dec. 19, 2005.

(51) Int. Cl. A61M 25/10

(52) U.S. Cl. 604/506; 604/103.01

(57) ABSTRACT

An expandable drug delivery device that can be implanted or otherwise delivered in and/or adjacent to a bone and/or soft tissue (e.g., connective tissue) for orthopedic applications is disclosed. Devices and methods are described herein for delivering agents for orthopedic and other uses. In particular such devices and methods can be useful for delivering agents to heal damaged tissue or prior to more invasive and traumatic orthopedic procedures.

### CROSS-REFERENCE TO RELATED APPLICATION

This application is a continuation of PCT International Application No. PCT/US2006/062337, filed Dec. 19, 2006 which claims the benefit of U.S. Provisional Application No. 60/751,882, filed Dec. 19, 2005, which are both incorporated herein by reference in their entireties.

### BACKGROUND OF THE INVENTION

This invention relates to devices and methods for delivering agents for orthopedic and other uses. In particular such devices and methods are useful in delivering agents to heal damaged tissue or prior to more invasive and traumatic orthopedic procedures. The invention includes use of a drug delivery device that is implanted or otherwise delivered in and/or adjacent to a bone and/or other soft tissue or connective tissue.

### BRIEF SUMMARY OF THE INVENTION

The invention includes methods and devices for providing a expandable delivery device that is implanted in bone and/or soft tissue in a minimally invasive manner and allows for delivery of various bioactive agents.

The expandable delivery device may comprise stents, anchors, or other support structures described herein. These expandable delivery devices can provide several functions such as: creating a support structure for damaged bone (fracture, tumor site. trauma, osteoporosis, osteonecrosis, etc.) in such case a filler may not be required to maintain support; creating a space in which substantial or sufficient amounts of filler and/or bioactive agents can be delivered into with capacitance (such that the healing response is improved over a duration of time); and/or delivery of a drug containing polymer designed to create a healing response for bone, cartilage, tendons, ligaments, joints, and/or joint resurfacing.

The term bioactive agent is meant to include any material that allows for an improvement in the rate of healing of damage tissue. For example, an agent may include cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof. Bioactive agents may also include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetlsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response.

### BRIEF DESCRIPTION OF THE DRAWINGS

FIG. 1 is a perspective view of a variation of the expandable delivery device.

FIG. 2 is a side view of the variation of the expandable delivery device of FIG. 1.

FIG. 3 is a top view of the variation of the expandable delivery device of FIG. 1.

FIG. 4 is a front view of the variation of the expandable delivery device of FIG. 1.

FIG. 5 is a perspective view of a variation of the expandable delivery device.

FIG. 6 is a side view of the variation of the expandable delivery device of FIG. 5.

FIG. 7 is a front view of the variation of the expandable delivery device of FIG. 5.

FIG. 8 is a perspective view of a variation of the expandable delivery device.

FIG. 9 is a front view of the variation of the expandable delivery device of FIG. 8.

FIG. 10 illustrates a flattened pattern for a variation of the expandable delivery device.

FIG. 11 is a perspective view of a variation of the expandable delivery device.

FIG. 12 is a front view of the variation of the expandable delivery device of FIG. 11.

FIG. 13 is a perspective view of a variation of the expandable delivery device.

FIG. 14 is a front view of the variation of the expandable delivery device of FIG. 13.

FIG. 15 is a perspective view of a variation of the expandable delivery device.

FIG. 16 is top view of the variation of the expandable delivery device of FIG. 15.

FIG. 17 is a side view of the variation of the expandable delivery device of FIG. 15.

FIG. 18 is a front view of the variation of the expandable delivery device of FIG. 1S.

FIG. 19 illustrates a variation of section A-A of the variation of the expandable delivery device of FIG. 15.

FIG. 20 illustrates a variation of section B-B of the variation of the expandable delivery device of FIG. 15.

FIG. 21 is a perspective view of a variation of the expandable delivery device.

FIG. 22 is top view of the variation of the expandable delivery device of FIG. 15.

FIG. 23 is a front view of the variation of the expandable delivery device of FIG. 1S.

FIGS. 24 and 25 illustrate a variation of a method for using a delivery system for the expandable support element.

FIGS. 26 through 28 illustrate a variation of a method for accessing a damage site in the vertebra.

FIG. 29 illustrates various variations of methods for deploying the expandable delivery device to the vertebral column.

FIGS. 30 through 32 illustrate a variation of a method for deploying the expandable delivery device into the damage site in the vertebra

FIGS. 33 and 34 illustrate a variation of a method for deploying the expandable delivery device into the damage site in the vertebra

FIGS. 35 and 36 illustrate a variation of a method for deploying one or more expandable delivery devices into one or more damage sites in the vertebra.

FIG. 37 illustrates a variation of a method for deploying the expandable delivery device into the damage site in the vertebra.

FIG. 38 illustrate a variation of a method for deploying the expandable delivery device into the damage site in the vertebra.

FIG. 39 illustrates variations of methods for deploying the expandable delivery device into the damage site in the vertebra.

FIGS. 40 and 41 illustrate a variation of a method for deploying the expandable delivery device into the damage site in the vertebra.

FIGS. 42 and 43 illustrate a variation of a method for deploying a locking pin into the expandable delivery device in the damage site in the vertebra.

FIGS. 44 through 49 illustrate a variation of a method for deploying a locking pin into the expandable delivery device.

FIG. 50 illustrates a variation of the buttress.

FIGS. 51 through 53 illustrate various variations of section C-C of the buttress of FIG. 50.

FIGS. 54 through 56 illustrate a variation of a method for deploying the buttress.

FIG. 57 illustrates a variation of a method for deploying the buttress.

FIGS. 58 through 60 illustrate a variation of a method for deploying the buttress

FIG. 61 illustrates a variation of the buttress.

FIG. 62 illustrates a variation of section D-D of the buttress of FIG. 61.

FIG. 63 illustrates a variation of a method for deploying the buttress.

FIGS. 64 through 67 illustrate a method for deploying the expandable delivery device of FIGS. 1 through 4.

FIGS. 68 through 70 illustrate a method for deploying the expandable delivery device of FIGS. 15 through 18.

FIG. 71 illustrates the deployed expandable delivery device of FIGS. 15 through 18 in use.

FIGS. 72 and 73 illustrate a method for deploying the expandable delivery device of FIGS. 19 and 20.

FIG. 74 illustrates a method of using the expandable deliver, device of FIGS. 15 through 18 with the band.

FIGS. 75 through 77 illustrate various variations of the locking pin.

FIG. 78 illustrates a variation of a method of using the delivery device in a femur.

FIG. 79a illustrates a variation of a method of using the delivery device to anchor soft tissue to hard tissue (e.g., tendon to bone).

FIG. 79b illustrates a variation of cross-section E-E of FIG. 79a

FIG. 80 illustrates a variation of a method of using the delivery device to anchor soft-tissue to soft tissue (e.g., a first ligament section to a second ligament section).

FIG. 81 illustrates a variation of a method of using the delivery device to anchor soft tissue to hard tissue (e.g., ligament to bone).

FIG. 82 illustrates a variation of a transverse cross-section of the delivery device of FIG. 81.

### DETAILED DESCRIPTION

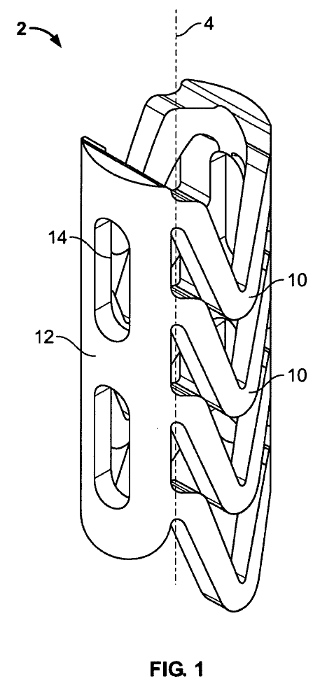

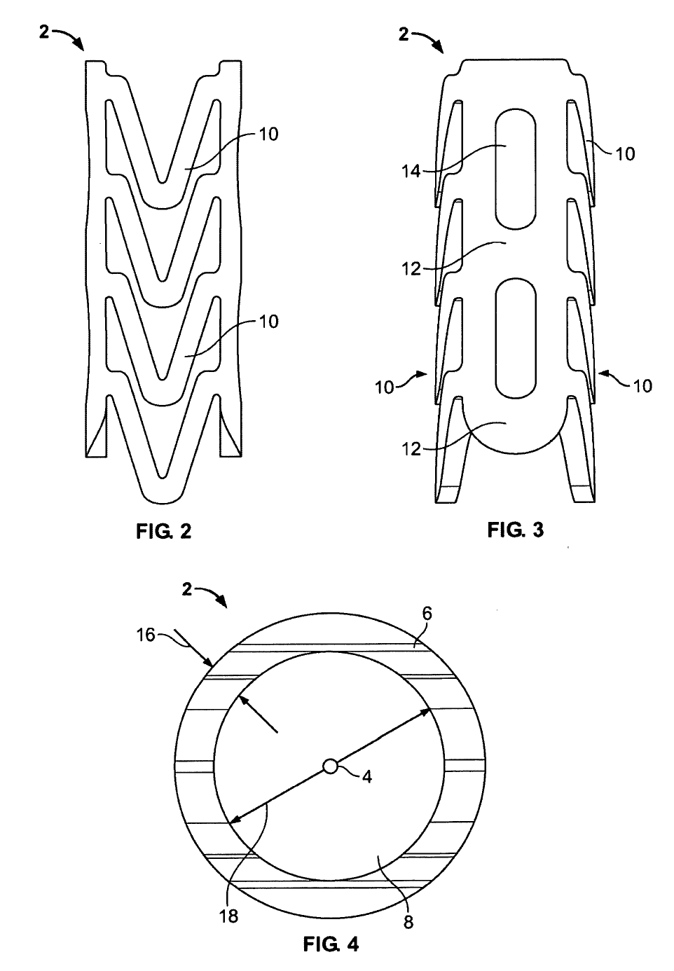

FIGS. 1 through 4 illustrate an biocompatible implant that can be used for tissue repair, for example for repair bone fractures such as spinal compression fractures, and/or repairing soft tissue damage, such as herniated vertebral discs. The implant can be an expandable delivery device 2, for example a stent. The expandable delivery device 2 can have a longitudinal axis 4. The expandable delivery device 2 can have an elongated wall 6 around the longitudinal axis 4. The expandable delivery device 2 can have a substantially and/or completely hollow longitudinal channel 8 along the longitudinal axis 4.

The wall 6 can have one or more first struts 10. The first struts 10 can be configured to be deformable and/or expandable. The wall 6 can have can have one or more second struts 12. The second struts 12 can be substantially undeformable and substantially inflexible. The first struts 10 can be flexibly (e.g., deformably rotatably) attached to the second struts 12.

The wall 6 can be configured to expand radially away from the longitudinal axis 4, for example in two opposite radial directions. A first set of first struts 10 can be aligned parallel to each other with respect to the longitudinal axis 4. A second set of first struts 10 can be aligned parallel to each other with respect to the longitudinal axis 4. The second set of first struts 10 can be on the opposite side of the longitudinal axis 4 from the first set of first struts 10. The second struts 12 can attached any or all sets of first struts 10 to other sets of first struts 10.

The second struts 12 can have one or more ingrowth ports. The ingrowth ports 14 can be configured to encourage biological tissue ingrowth therethrough during use. The ingrowth ports 14 can be configured to releasably and/or fixedly attach to a deployment tool or other tool. The ingrowth ports 14 can be configured to increase, and/or decrease, and/or focus pressure against the surrounding biological tissue during use. The ingrowth ports 14 can be configured to increase and/or decrease the stiffness of the second struts 12. The ingrowth ports 14 can be configured to receive and/or attach to a buttress.

The first struts 10 can be configured to have a “V” shape. The space between adjacent first struts 10 can be configured to receive and/or attach to a locking pin during use.

The wall 6 can have a wall thickness 16. The wall thickness 16 can be from about 0.25 mm (0.098 in.) to about 5 mm (0.2 in.), for example about 1 mm (0.04 in.).

The wall 6 can have an inner diameter 18. The inner diameter 18 can be from about 1 mm (0.04 in.) to about 30 mm (1.2 in.), for example about 6 mm (0.2 in.). The wall thickness 16 and/or the inner diameter 18 can vary with respect to the length along the longitudinal axis 4. The wall thickness 16 and/or the inner diameter 18 can vary with respect to the angle formed with a plane parallel to the longitudinal axis 4.

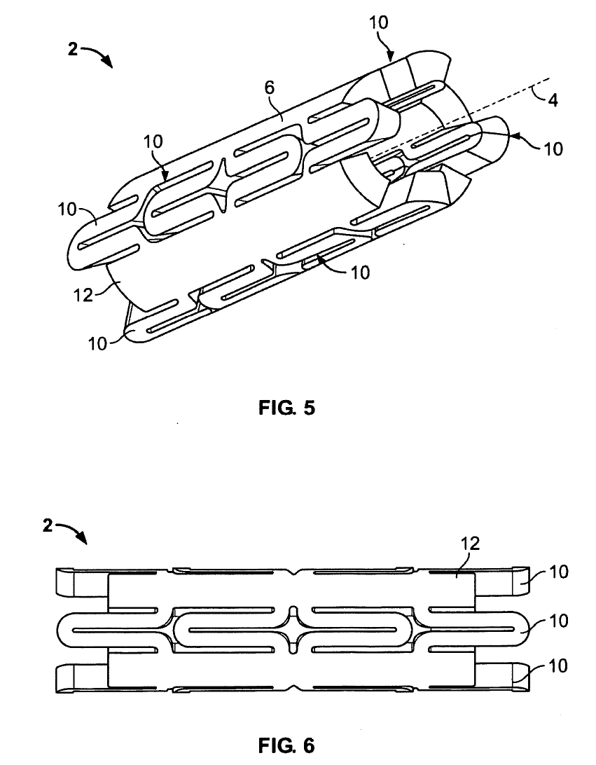

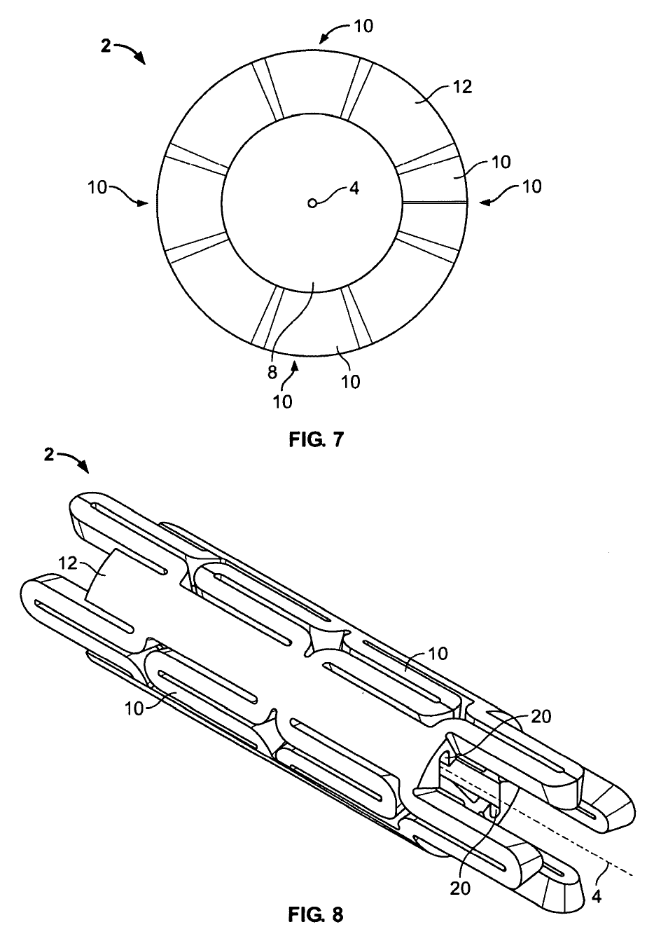

FIGS. 5 through 7 illustrate an expandable delivery device 2 that can be configured to expand away from the longitudinal axis 4 in more than two opposite directions, for example in two sets of two opposite radial directions. The wall 6 can have four sets of first struts 10. Each set of first struts 10 can be opposite to another set of first struts 10, radially with respect to the longitudinal axis 4. Each of four sets of second struts 12 can attach each set of first struts 10.

The first struts 10 on a first longitudinal half of the expandable delivery device can be oriented (e.g., the direction of the pointed end of the “V” shape) in the opposite direction as the first struts 10 on a second longitudinal half of the expandable delivery device 2.

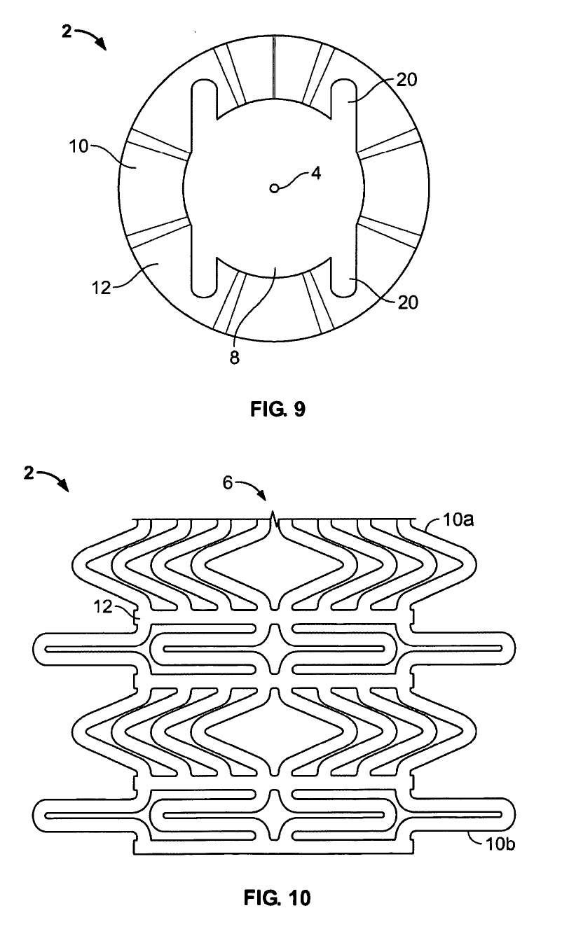

FIGS. 8 and 9 illustrate that the longitudinal channel 8 can have one or more lock grooves 20. The lock grooves 20 can be configured to receive and/or slidably and fixedly or releasably attach to a locking pin.

FIG. 10 illustrates a visually flattened pattern of the wall 6 for the expandable delivery device 2. (The pattern of the wall 6 can be flattened for illustrative purposes only, or the wall 6 can be flattened during the manufacturing process.) The pattern can have multiple configurations for the first and/or second struts 10 and/or 12. For example, first struts 10a can have a first configuration (e.g., a “V” shape) and first struts 10b can have a second configuration (e.g., a “U” shape).

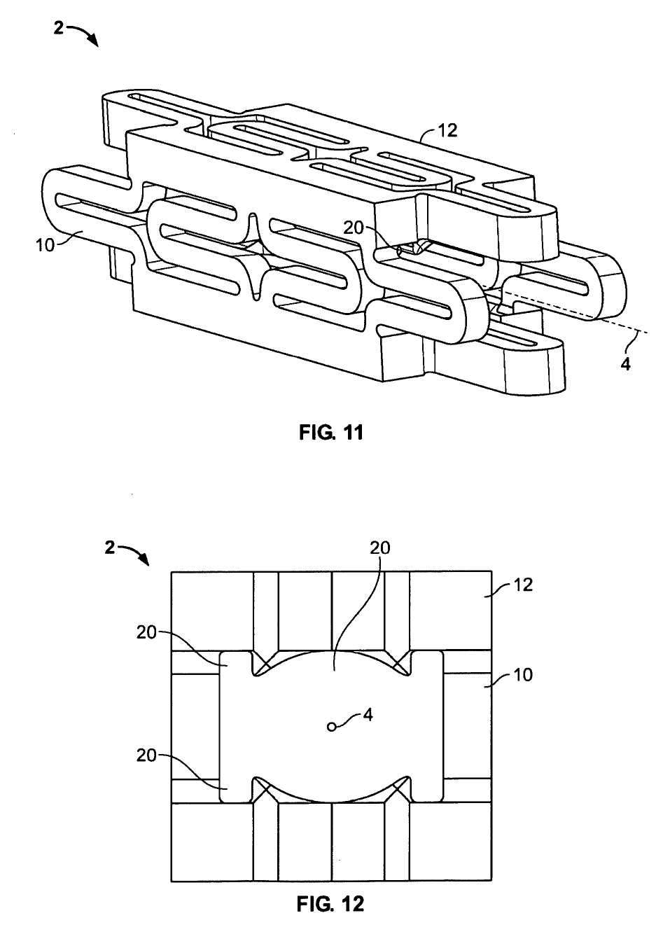

FIGS. 11 and 12 illustrate that the expandable delivery device 2 can have a square, rectangular, circular (shown elsewhere), oval (not shown) configuration or combinations thereof (e.g., longitudinal changes in shape).

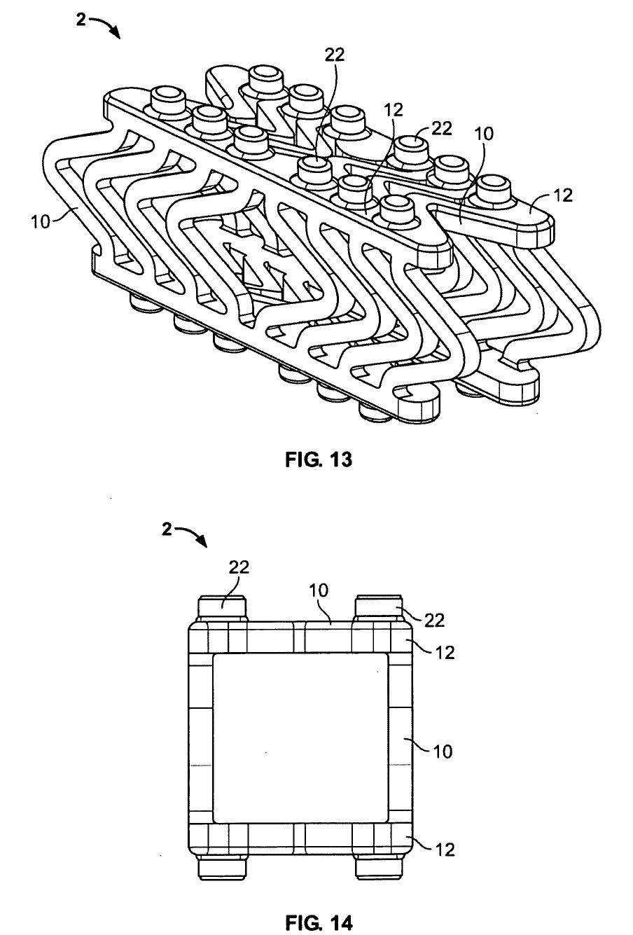

FIGS. 13 and 14 illustrate that the expandable delivery device 2 can have protruding tissue engagement elements, such as tissue hooks, and/or barbs, and/or cleats 22. The cleats 22 can be integral with and/or fixedly or removably attached to the first and/or second struts 12. The cleats 22 can be on substantially opposite sides of the expandable delivery device 2.

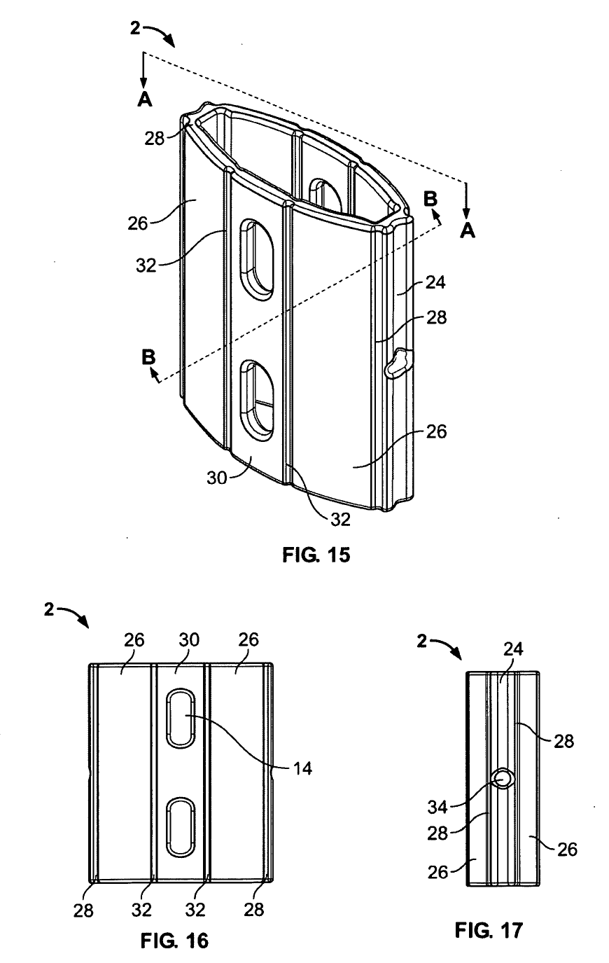

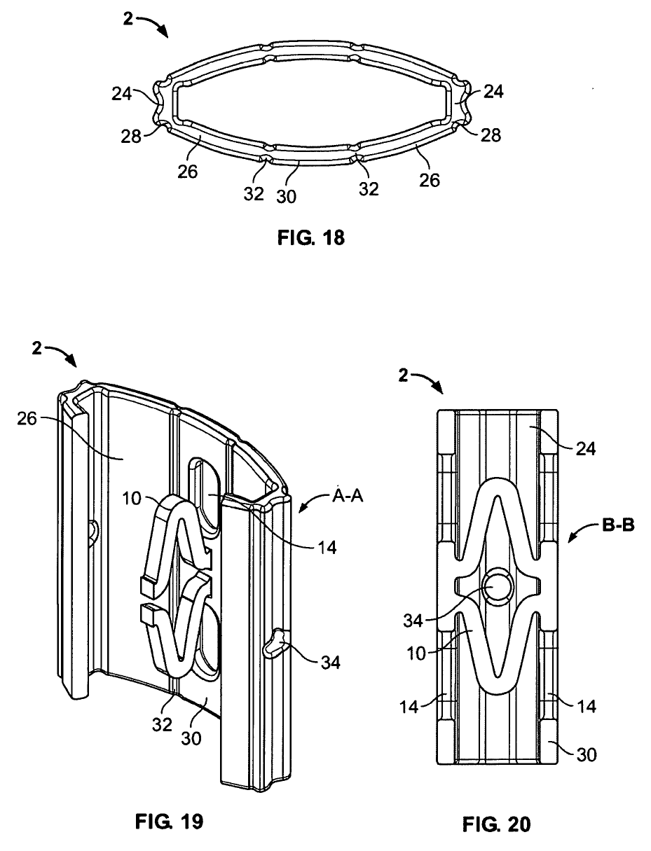

FIGS. 15 through 18 illustrate that the expandable delivery device 2 can have panels attached to other panels at flexible joints. The expandable delivery device 2 can have first panels 24 attached to and/or integral with second panels 26 at first joints The second panels 26 can be attached to and/or integral with third panels 30 at second joints 32. The expandable delivery device 2 can have one or more tool engagement ports 34, for example on the first panels 24. The expandable delivery device 2 can have one or more ingrowth ports 14, for example, on the third panels 30. The outside of the first panel 24 can be concave.

FIGS. 19 and 20 illustrate that the expandable delivery device 2 can have first and/or second struts 10 and/or 12 and panels. The first and/or second struts 10 and/or 12 can be internal to the panels. The first struts 10 can be attached to the third panels 30.

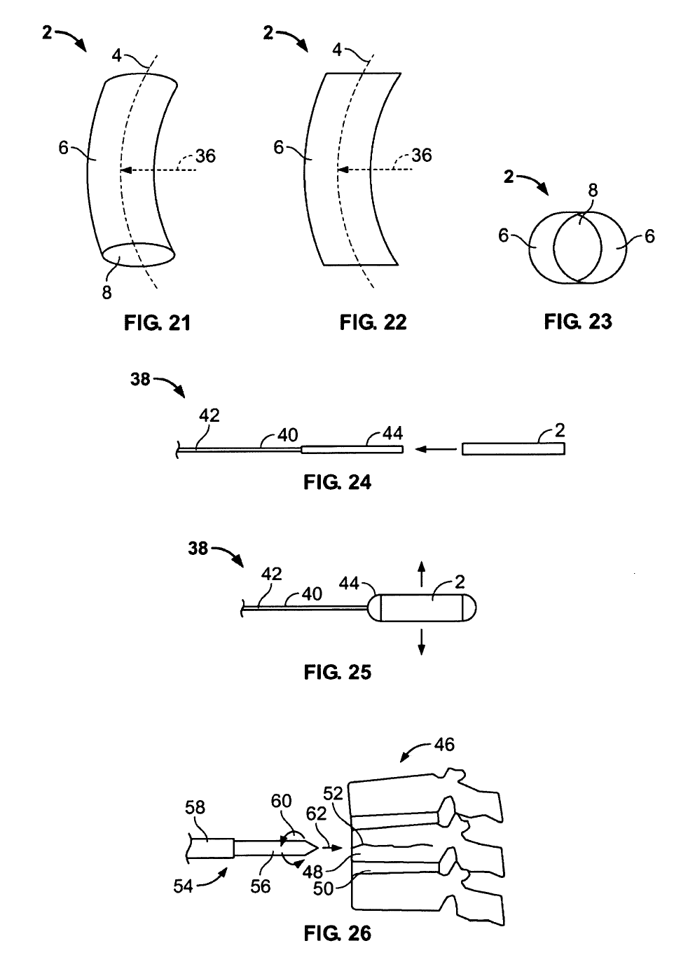

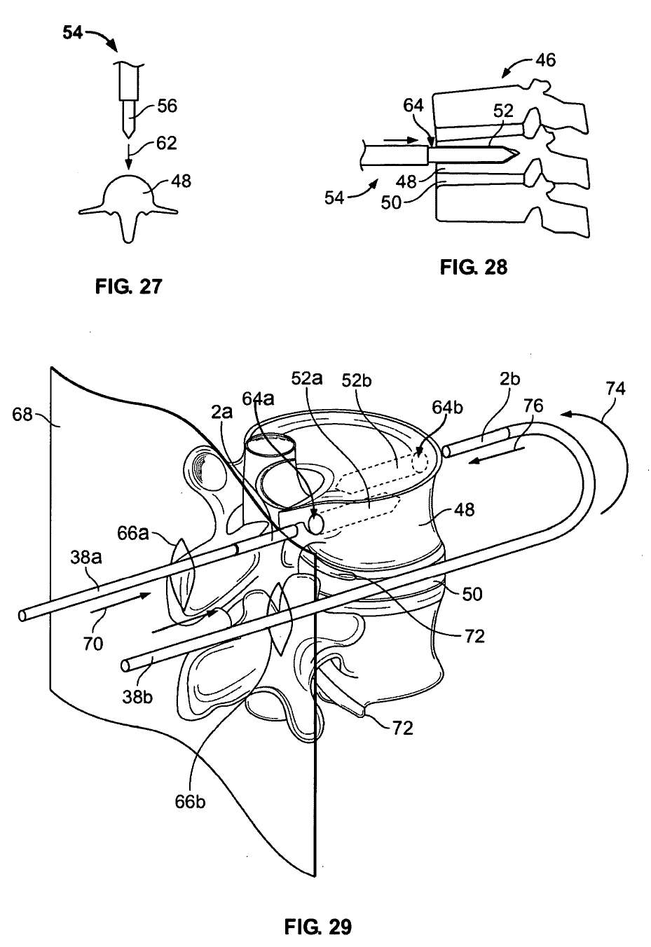

FIGS. 21 through 23 illustrate the expandable delivery device 2 that can have a radius of curvature 36 along the longitudinal axis 4. The radius of curvature 36 can be from about 1 mm (0.04 in.) to about 250 mm (10 in.), for example about 50 mm (2 in.). (The wall 6 is shown sans panels or struts for illustrative purposes.) The expandable delivery device 2 can have at least one flat side, for example two flat sides. The two flat sides can be on opposite sides of the expandable delivery device 2 from each other.

Variations of the expandable delivery devices (including those labeled as expandable support devices) and methods of use, and tools for deployment are disclosed in the following applications, all of which are incorporated by reference herein in their entireties: PCT application No. PCT/US05/034115, filed 21 Sep. 14, 2005; U.S. Provisional Application No. 60/675,512, filed Apr. 27, 2005; U.S. Provisional Application No. 60/699,577, filed Jul. 14, 2005; U.S. Provisional Application No. 60/699,576, filed Jul. 14, 2005; U.S. Provisional Patent Application No. 60/675,543, filed 27 Apr. 2005; PCT Application No. PCT/US2005/034742, filed 26 Sep. 18, 2005; PCT Application No. PCT/US2005/034728, filed 26 Sep. 2005; PCT Application No. PCT/US2005/037126, filed 12 Oct. 2005; U.S. Provisional Patent Application No. 60/723,309, filed 4 Oct. 2005; U.S. Provisional Patent Application No. 60/675,512, filed 27 Apr. 2005; and U.S. Provisional Patent Application No. 60/699,577, filed 14 Jul. 2005.

FIG. 24 illustrates that the expandable delivery device 2 can be loaded in a collapsed (i.e., contracted) configuration onto a deployment tool 38. The deployment tool 38 can have an expandable balloon catheter as known to those having an ordinary level of skill in the art. The deployment tool 38 can have a catheter 40. The catheter 40 can have a fluid conduit 42. The fluid conduit 42 can be in fluid communication with a balloon 44. The balloon 44 and the deployment tool 38 can be the balloon 44 and deployment tool 38, for example, as described by PCT Application No. PCT/US2005/033965, filed 21 Sep. 2005; PCT Application No. PCT/US2006/061438, filed 30 Nov. 2006; U.S. Provisional Application No. 60/611,972; filed 21 Sep. 2004; and U.S. Provisional Application No. 60/740,792, filed 30 Nov. 2005, which are all herein incorporated by reference in their entireties. The balloon 44 can be configured to receive a fluid pressure of at least about 5,000 kPa (50 atm), more narrowly at least about 10,000 kPa (100 atm), for example at least about 14,000 kPa (140 atm).

The deployment tool 38 can be a pair of wedges, an expandable jack, other expansion tools, or combinations thereof.

FIG. 25 illustrates that the fluid pressure in the fluid conduit 42 and balloon can increase, thereby inflating the balloon 44, as shown by arrows. The expandable delivery device 2 can expand, for example, due to pressure from the balloon 44.

FIGS. 26 (side view) and 27 (top view) illustrates a vertebral column 46 that can have one or more vertebra 48 separated from the other vertebra 48 by discs 50. The vertebra 48 can have a damage site 52, for example a compression fracture.

An access tool 54 can be used to gain access to the damage site 52 and or increase the size of the damage site 52 to allow deployment of the expandable delivery device 2. The access tool 54 can be a rotating or vibrating drill 56 that can have a handle 58. The drill 56 can be operating, as shown by arrows 60. The drill 56 can then be translated, as shown by arrow 62, toward and into the vertebra 48 so as to pass into the damage site 52.

FIG. 28 illustrates that the access tool 54 can be translated, as shown by arrow, to remove tissue at the damage site 52. The access tool 54 can create an access port 64 at the surface of the vertebra 48. The access port 64 can open to the damage site 52. The access tool 54 can then be removed from the vertebra 48.

FIG. 29 illustrates that a first deployment system 38a can enter through the subject's back. The first deployment system 38a can enter through a first incision 66a in skin 68 on the posterior side of the subject near the vertebral column 46. The first deployment system 38a can be translated, as shown by arrow 70, to position a first expandable delivery device 2a into a first damage site 52a. The first access port 64a can be on the posterior side of the vertebra 48.

A second deployment system 38b can enter through a second incision 66b (as shown) in the skin 68 on the posterior or the first incision 66a. The second deployment tool 38b can be translated through muscle (not shown), around nerves 72, and anterior of the vertebral column 46. The second deployment system 38b can be steerable. The second deployment system 38b can be steered, as shown by arrow 74, to align the distal tip of the second expandable delivery device 2b with a second access port 64b on a second damage site 52b. The second access port 64b can face anteriorly. The second deployment system 38b can translate, as shown by arrow 76, to position the second expandable delivery device 2 in the second damage site 52b.

The vertebra 48 can have multiple damage sites 52 and expandable delivery devices 2 deployed therein. The expandable delivery devices 2 can be deployed from the anterior, posterior, both lateral, superior, inferior, any angle, or combinations of the directions thereof.

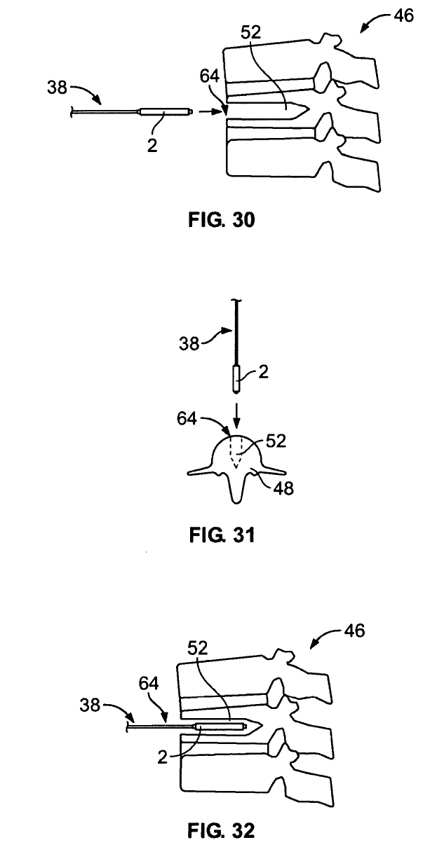

FIGS. 30 and 31 illustrate translating, as shown by arrow, the deployment tool 38 loaded with the expandable delivery device 2 through the access port 64. FIG. 32 illustrates locating the expandable delivery device 2 on the deployment tool in the damage site 52.

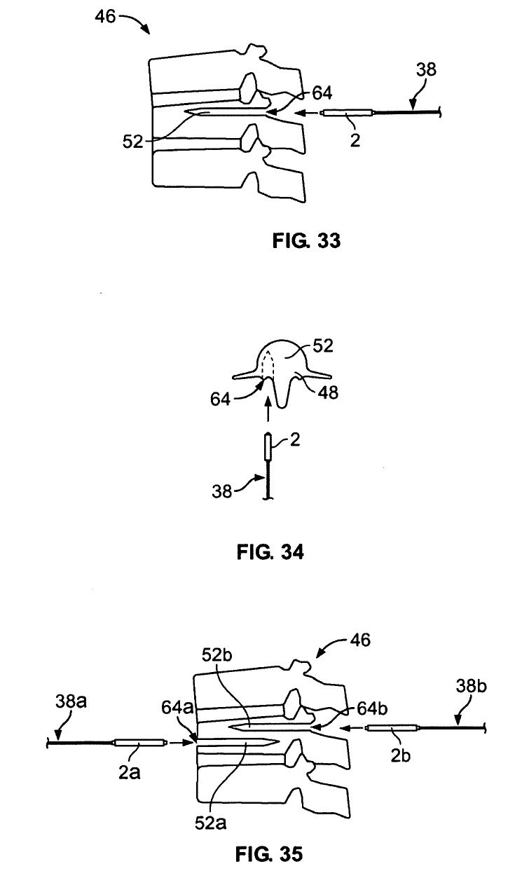

FIGS. 33 and 34 illustrate that the deployment tool 38 can be deployed from the posterior side of the vertebral column 46. The deployment tool 38 can be deployed off-center, for example, when approaching the posterior side of the vertebral column 46.

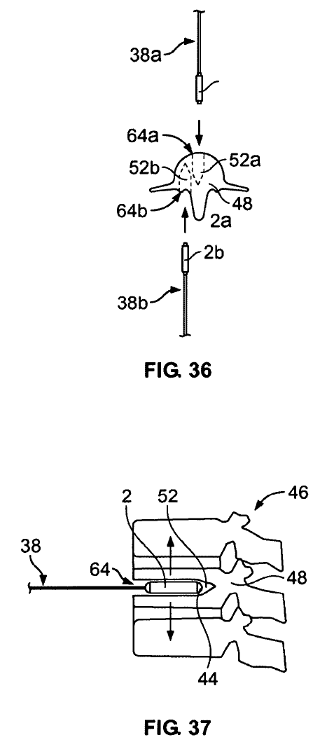

FIGS. 35 and 36 illustrate that first and second deployment tools 38a and 38b can position and deploy first and second expandable delivery devices 2a and 2b simultaneously, and/or in the same vertebra 48 and into the same or different damage sites 52a and 52b.

FIG. 37 illustrates that the fluid pressure in the fluid conduit 42 and the balloon 44 can increase, thereby inflating the balloon 44, as shown by arrows. The expandable delivery device 2 can expand, for example, due to pressure from the balloon 44. The balloon 44 can be expanded until the expandable delivery device 2 is substantially fixed to the vertebra 48. The balloon 44 and/or the expandable delivery device 2 can reshape the vertebral column 46 to a more natural configuration during expansion of the balloon 44.

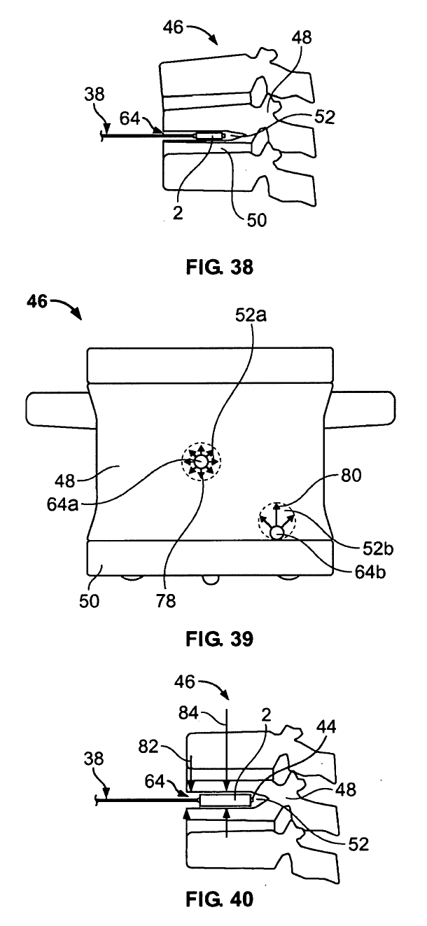

FIG. 38 illustrates that the access port 64 can be made close to the disc 50, for example when the damage site 52 is close to the disc 50. The deployment tool 38 can be inserted through the access port 64 and the expandable delivery device 2 can be deployed as described supra

FIG. 39, a front view of the vertebral column, illustrates that more than one expandable delivery device 2 can be deployed into a single vertebra 48. For example, a first expandable delivery device (not shown) can be inserted through a first access port 64a and deployed in a first damage site 52a, and a second expandable delivery device (not shown) can be inserted through a first access port 64a and deployed in a second damage site 52b.

The first access port 64a can be substantially centered with respect to the first damage site 52a. The first expandable delivery device (not shown) can expand, as shown by arrows 78, substantially equidirectionally, aligned with the center of the first access port 64a The second access port 64b can be substantially not centered with respect to the second damage site 52b. The second expandable delivery device (not shown) can substantially anchor to a side of the damage site 52 and/or the surface of the disc 50, and then expand, as shown by arrows 80, substantially directionally away from the disc 50.

FIG. 40 illustrates that the fluid pressure can be released from the balloon 44, and the balloon 44 can return to a pre-deployment configuration, leaving the expandable support element substantially fixed to the vertebra 48 at the damage site 52.

The access port 64 can have an access port diameter 82. The access port diameter 82 can be from about 1.5 mm (0.060 in.) to about 40 mm (2 in.), for example about 8 mm (0.3 in.). The access port diameter 82 can be a result of the size of the access tool 54. After the expandable delivery device 2 is deployed, the damage site can have a deployed diameter 84. The deployed diameter 84 can be from about 1.5 mm (0.060 in.) to about 120 mm (4.7 in.), for example about 20 mm (0.8 in.). The deployed diameter 84 can be greater than, equal to, or less than the access port diameter 82.

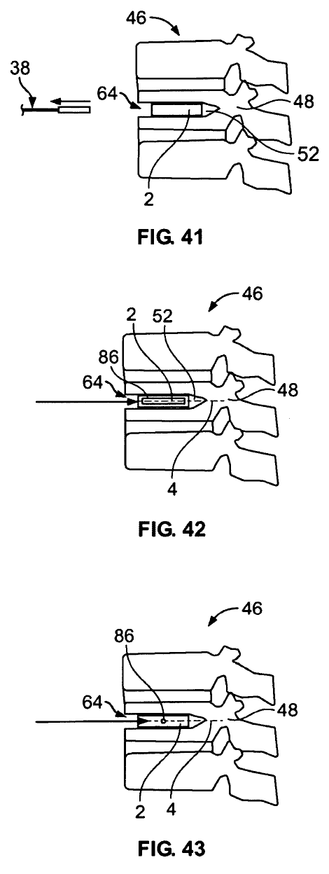

FIG. 41 illustrates that the deployment tool 38 can be removed, as shown by arrow, from the vertebra 48 after the expandable delivery device 2 is deployed.

FIGS. 42 and 43 illustrate that a locking pin 86 can be inserted, as shown by arrow, into the deployed expandable delivery device 2, for example, after the expandable delivery device 2 is deployed in the vertebra 48. The locking pin 86 can prevent the expandable delivery device 2 from collapsing after the expandable delivery device 2 is deployed in the vertebra 48. The locking pin 86 can form an interference fit with the expandable delivery device 2.

The locking pin 86 can be parallel with the longitudinal axis 4, as shown in FIG. 42, for example when the locking pin 86 is slidably received by and/or attached to the lock grooves 20. The locking pin 86 can be perpendicular to the longitudinal axis 4, as shown in FIG. 43, for example when the locking pin 86 is slidably received by and/or attached to ports formed between adjacent first struts 10 after the expandable delivery device 2 is expanded.

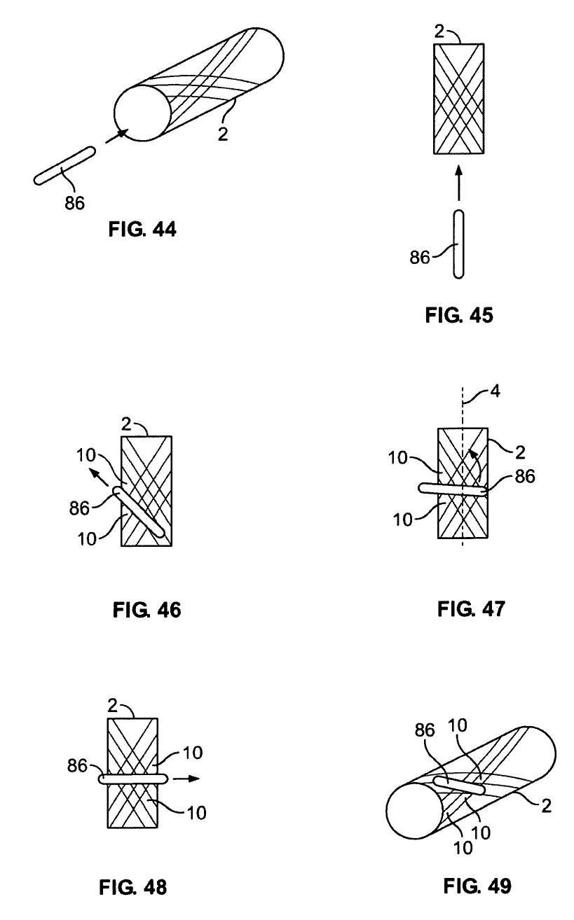

FIGS. 44 through 49 illustrate a method for deploying the locking pin 86 into the expandable delivery device 2. As shown in FIGS. 44 and 45, the locking pin 86 can be translated, as shown by arrow, into the expandable delivery device 2. As shown in FIG. 46, a first end of the locking pin 86 can be translated, as shown by arrow, into a first port formed between adjacent first struts 10. As shown by FIG. 47, a second end of the locking pin 86 can be rotated, as shown by arrow. As shown by FIG. 48, the second end of the locking pin 86 can be translated, as shown by arrow, into a second port formed between adjacent first struts 10. FIG. 49 shows the locking pin 86 deployed into, and forming an interference fit with, the expandable delivery device 2.

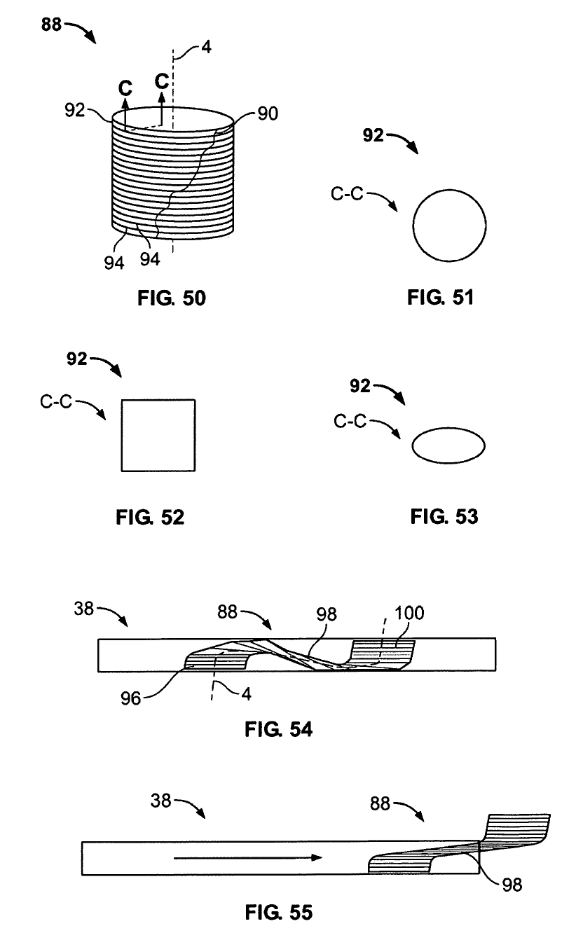

FIG. 50 illustrates a buttress 88. The buttress 88 can have a longitudinal axis 4. The buttress 88 can have a tensioner 90. A first end of the tensioner 90 can be fixedly or removably attached a first end of the buttress 88. A second end of the tensioner 90 can be fixedly or removably attached a second end of the buttress 88. The tensioner 90 can be in a relaxed configuration when the buttress 88 is in a relaxed configuration. The tensioner 90 can create a tensile force between the first end of the buttress 88 and the second end of the buttress 88 when the buttress 88 is in a stressed configuration. The tensioner 90 can be, for example, a resilient wire, a coil spring, an elastic member, or combinations thereof.

The buttress 88 can have a coil 92. The coil 92 can have turns 94 of a wire, ribbon, or other coiled element. FIGS. 51 through 53 illustrate that the coil can be made from a wire, ribbon, or other coiled element having a circular, square, or oval cross-section, respectively.

The buttress 88 can be a series of connected hoops.

FIG. 54 illustrates that the buttress 88 can be loaded into a hollow deployment tool 38 in a smear (i.e., partially shear stressed) configuration. The buttress 88 in the smear configuration can have a relaxed first end 96, a stressed smear section 98, and a relaxed second end 100. The longitudinal axis 4 can be not straight (i.e., non-linear) through the smear section 98.

FIG. 55 illustrates that part of the buttress 88 can be forced, as shown by arrow, out of the deployment tool 38. The second end 100 can exit the deployment tool 38 before the remainder of the buttress 88. The smear section 98 can then partially relax. The second end 100 can be positioned to a final location before the remainder of the buttress 88 is deployed from the deployment tool 38.

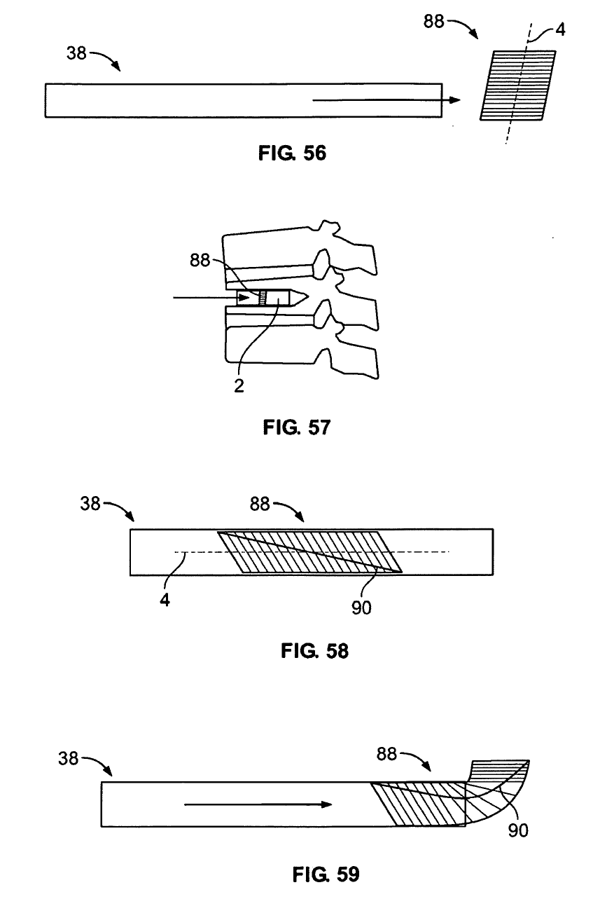

FIG. 56 illustrates that the remainder of the buttress 88 can be forced, as shown by arrow, out of the deployment tool 38. The smear section 98 can substantially relax. The longitudinal axis 4 can return to a substantially relaxed and/or straight (i.e., linear) configuration.

FIG. 57 illustrates that the buttress 88 can be deployed in the expandable delivery device 2, for example with the longitudinal axis 4 of the buttress 88 or the strongest orientation of the buttress 88 aligned substantially parallel with the primary load bearing direction (e.g., along the axis of the spine) of the expandable delivery device 2.

FIG. 58 illustrates that the buttress 88 can be loaded into the hollow deployment tool 38 with the longitudinal axis 4 of the buttress 88 substantially parallel with the hollow length of the deployment tool 38. The entire length of the buttress 88 can be under shear stress.

FIG. 59 illustrates that part of the buttress 88 can be forced, as shown by arrow, out of the deployment tool 38. The second end of the buttress 88 can exit the deployment tool 38 before the remainder of the buttress 88. The tensioner 90 can apply a tensile stress between the ends of the buttress 88, for example, forcing the deployed second end of the buttress 88 to “stand up straight”. The second end of the buttress 88 can be positioned to a final location before the remainder of the buttress is deployed from the deployment tool 38.

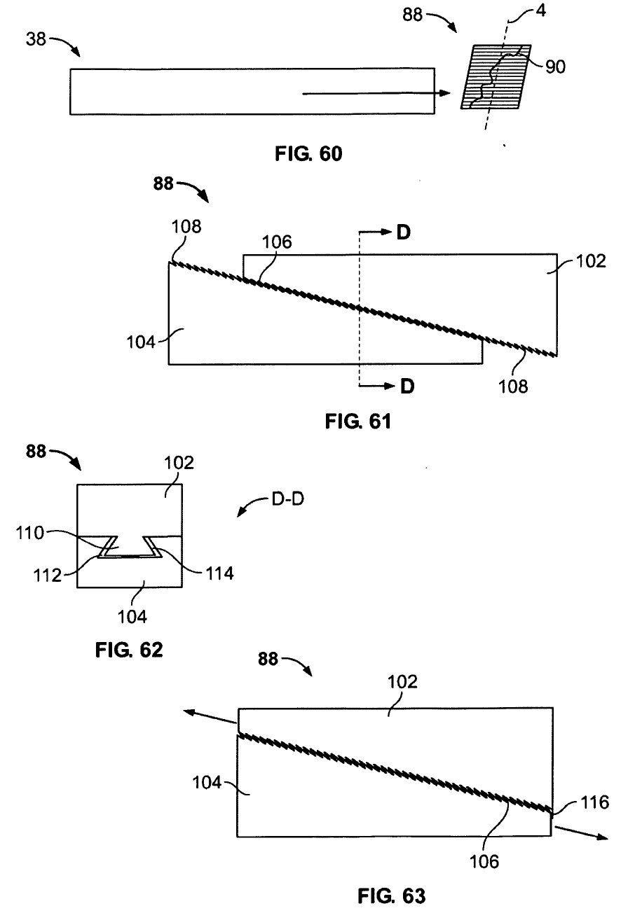

FIG. 60 illustrates that the remainder of the buttress 88 can be forced, as shown by arrow, out of the deployment tool 38. The buttress 88 can substantially relax.

FIG. 61 illustrates that the buttress can have a first wedge 102 and a second wedge 104. The first wedge 102 can contact the second wedge 104 at a directionally locking interface 106. The directionally locking interface 106 can have directional teeth 108.

FIG. 62 illustrates that the first wedge 102 can be slidably attached to the second wedge 104. The first wedge 102 can have a tongue 110. The second wedge 104 can have a groove 112. The tongue 110 can be slidably attached to the groove 112.

A gap 114 can be between the tongue 110 and the groove 112. The gap 114 can be wider than the height of the teeth 108. The gap 114 can be configured to allow the first wedge 102 to be sufficiently distanced from the second wedge 104 so the teeth 108 on the first wedge 102 can be disengaged from the teeth 108 on the second wedge 104.

The buttress 88 in a compact configuration can be placed inside of the longitudinal channel 8 of the deployed expandable delivery device 2. FIG. 63 illustrates that the first wedge 102 can then be translated, as shown by arrows, relative to the second wedge 104 along the directionally locking interface 106. The first wedge 102 can abut a first side of the inside of the deployed expandable delivery device 2. The second wedge 104 can abut a second side of the inside of the deployed expandable delivery device 2. The directionally interference fitting teeth 108 can prevent disengagement of the buttress 88. A stop 116 can limit the relative translation of the first wedge 102 and the second wedge 104.

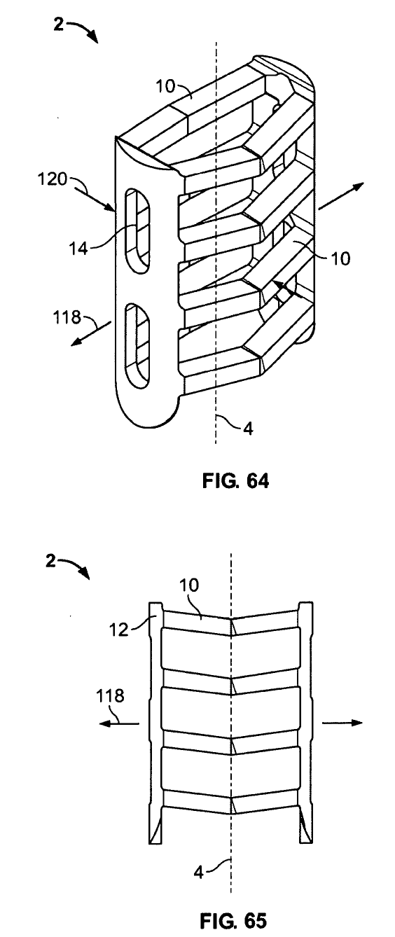

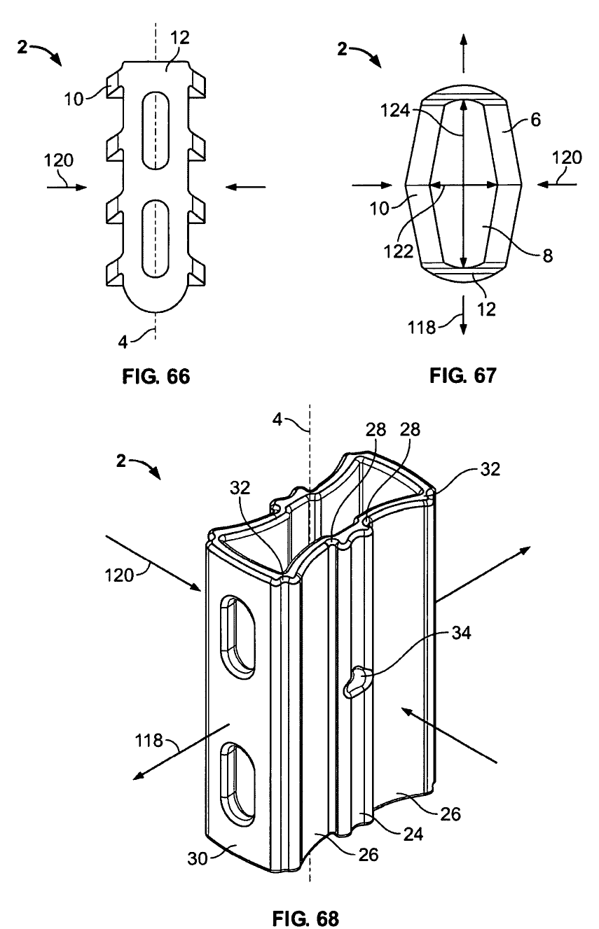

FIGS. 64 through 67 illustrate the expandable delivery device 2 of FIGS. 1 through 4 that can be in a deployed configuration. The first struts 10 can be expanded, as shown by arrows 118. The expandable delivery device 2 can passively narrow, as shown by arrows 120. The expandable delivery device 2 can be deployed in a configuration where the second struts 12 can be placed against the load bearing surfaces of the deployment site.

The expandable delivery device 2 can have a minimum inner diameter 122 and a maximum inner diameter 124. The minimum inner diameter 122 can be less than the pre-deployed inner diameter. The minimum inner diameter 122 can be from about 0.2 mm (0.01 in.) to about 120 mm (4.7 in.), for example about 2 mm (0.08 in.) be from about 1.5 mm (0.060 in.) to about 40 mm (2 in.), for example about 8 mm (0.3 in.). The maximum inner diameter 124 can be more than the pre-deployed inner diameter. The maximum inner diameter 124 can be from about 1.5 mm (0.060 in.) to about 120 mm (4.7 in.), for example about 18 mm (0.71 in.).

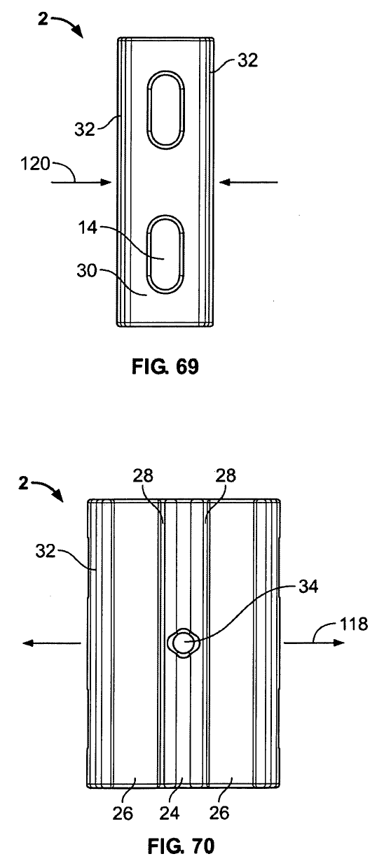

FIGS. 68 through 70 illustrate the expandable delivery device 2 of FIGS. 15 through 18 that can be in a deployed configuration. A tool (not shown) can releasably attach to the tool engagement port 34. The tool can be used to position the expandable delivery device 2. The tool can be used to expand the expandable delivery device 2, for example, by forcing the first panels 24 toward each other.

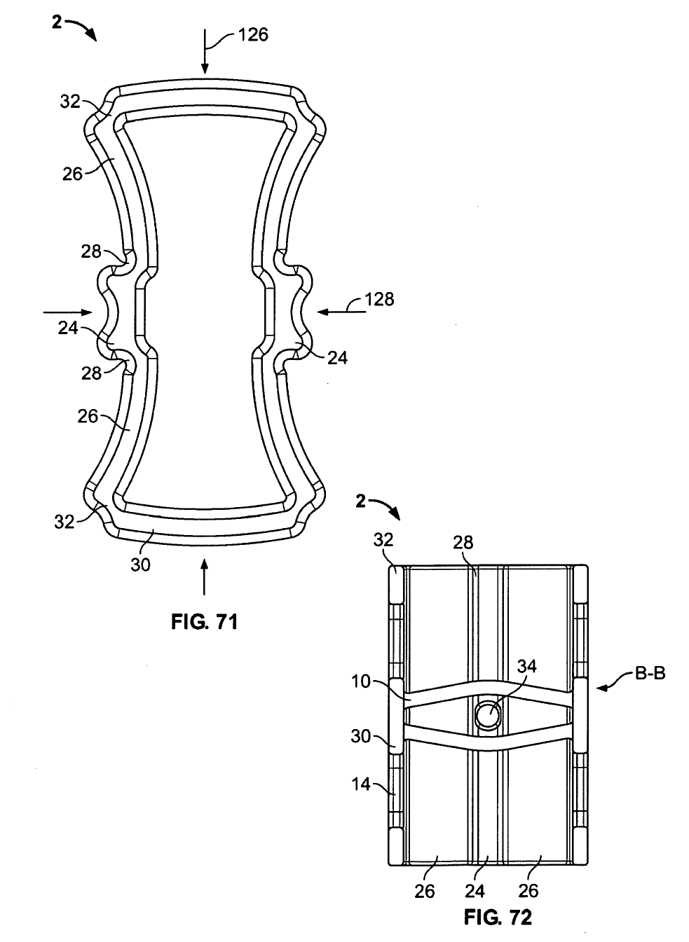

The second joints 32 can form angles less than about 90°. As shown in FIG. 71, a compressive force, as shown by arrows 126, causes additional inward deflection, as shown by arrows 128, of the first panels 24, and will not substantially compress the expandable delivery device 2.

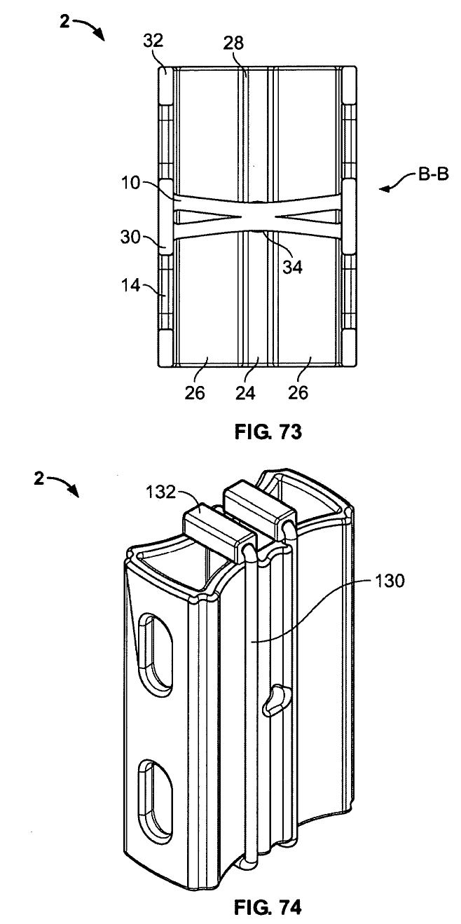

FIG. 72 illustrates a deployed configuration of the expandable delivery device 2 of FIGS. 19 and 20. The first struts 10 can expand to the size of the expandable delivery device 2. FIG. 73 illustrates that the first struts 10 can touch each other, for example if the expandable delivery device 2 is sufficiently expanded. In the case of extreme compressive loads applied to the expandable delivery device 2, the first struts 10 can buckle into each other, thereby providing additional resistance to compressive loads.

FIG. 74 illustrates the expandable delivery device 2 that can have one or more bands 130. The bands 130 can be attached to other bands 130 and/or attached to the expandable delivery device 2 with band connectors 132. The bands 130 can be attached to the expandable delivery device 2 before, during, or after deployment. The bands 130 can increase the compressive strength of the expandable delivery device 2.

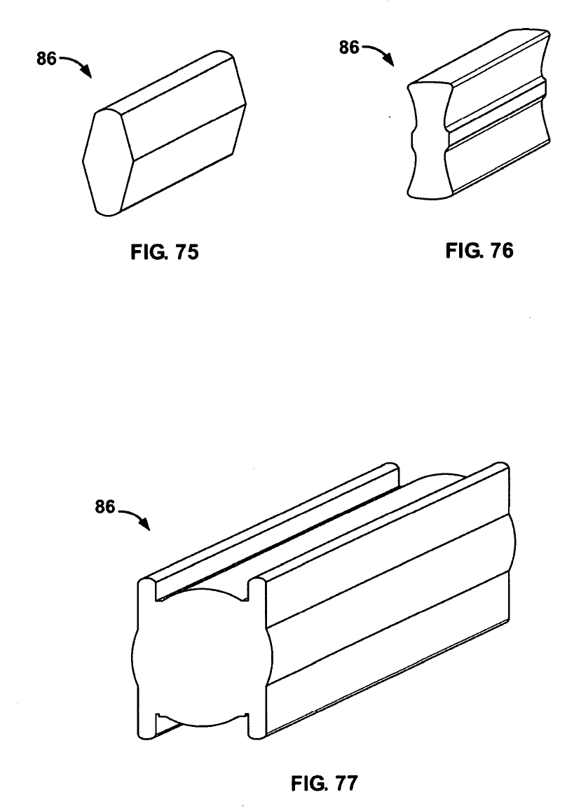

FIG. 75 illustrates the locking pin 86 that can be configured to fit into the longitudinal port 8, for example, of the expanded expandable delivery device 2 of FIGS. 64 through 67. FIG. 76 illustrates the locking pin 86 that can be configured to fit into the longitudinal port 8, for example, of the expanded expandable delivery device 2 of FIGS. 68 through 71. FIG. 77 illustrates the locking pin 86 that can be configured to fit into the longitudinal port 8, for example, of the expanded expandable delivery device 2 of FIGS. 8 and 9 and/or FIGS. 11 and 12.

Once the expandable delivery device 2 is deployed, the longitudinal channel 8 and the remaining void volume in the damage site 52 can be filled with, for example, biocompatible coils, bone cement, morselized bone, osteogenic powder, beads of bone, polymerizing fluid, paste, a matrix (e.g., containing an osteogenic agent and/or an anti-inflammatory agent, and/or any other agent disclosed supra), Orthofix, cyanoacrylate, or combinations thereof.

The expandable delivery device 2 can be implanted in the place of all or part of a vertebral disc 50. For example, if the disc 50 has herniated, the expandable delivery device 2 can be implanted into the hernia in the disc annulus, and/or the expandable delivery device 2 can be implanted into the disc nucleus.

As discussed above, the expandable delivery devices may act as expandable delivery devices that are implanted in bone and/or soft tissue in a minimally invasive manner and allows for delivery of various bioactive agents. It is noted that in any of the above examples, the expandable delivery device may be combined with bioactive agents or fillers to improve the healing response of the damaged tissue.

Once the device is expanded it creates instant support. In addition, the device can it will deliver a bioactive agent via a coating on the device or by creating a space ideal for packing the device with non hardening fillers such as bioactive agents and/or bone chips, ceramics, polymers, as described herein.

In order to create the ideal healing condition, the expandable member/expandable delivery device forms a structure upon deployment that results in fixation within the tissue. The device may be fabricated as discussed herein and may be either self expanding, balloon expanded, or mechanically expanded. The bioactive agents provide the biochemical accelerators used to promote healing, increase bone density, etc. The bioactive agents can be designed to release slowly over long periods in order to produce the needed healing effects for each particular application.

The expandable delivery device 2 can be inserted into a bone experiencing osteoporosis (e.g., that has lost normal density and as a result is fragile).

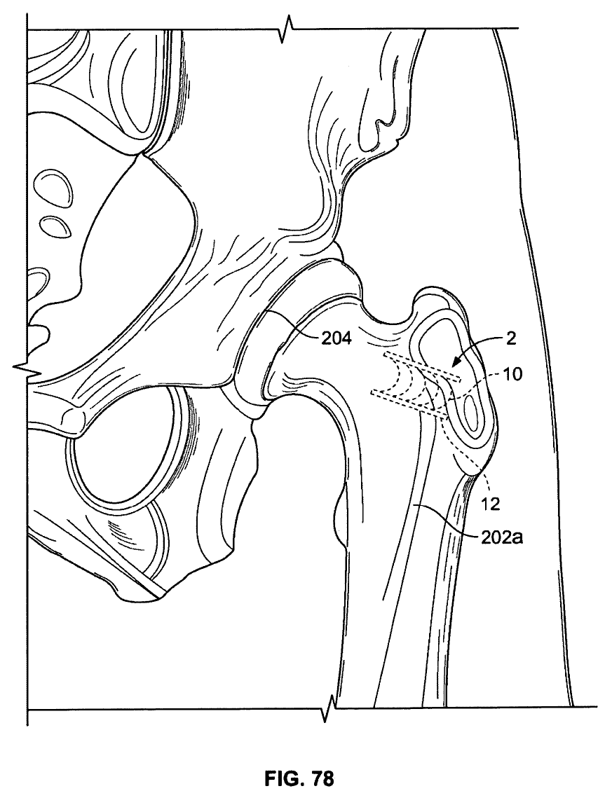

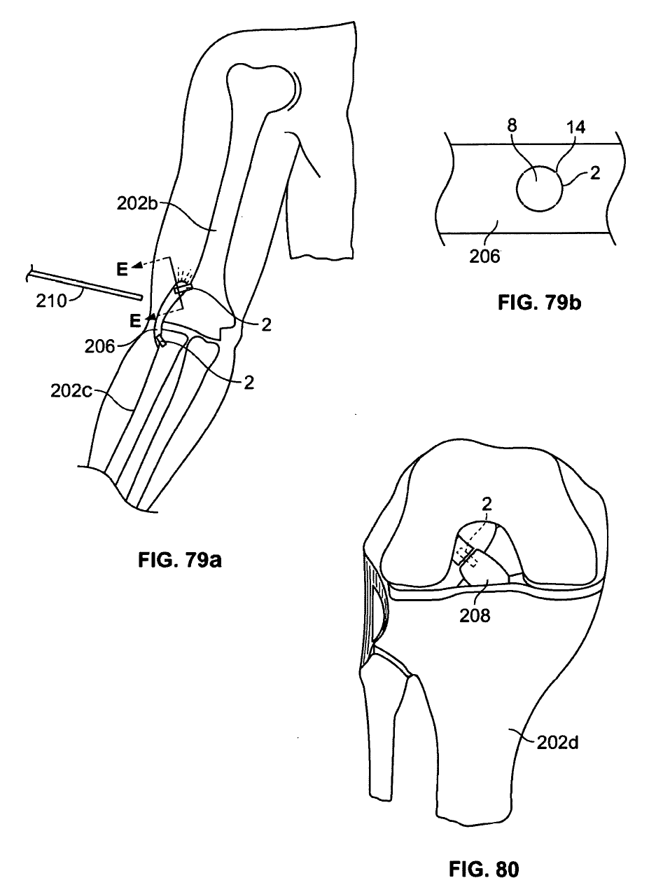

FIG. 78 illustrates that the expandable delivery device 2 may be placed in a femur, for example at the hip. This can be before or after the need for a hip replacement is diagnosed and/or performed. For example, the expandable support device 2 can be used as a femoral stem or anchor for a total hip replacement prosthesis, or as a collar for a femoral stem of a total hip replacement prosthesis. The delivery device can be implanted in any long bone, for agent delivery and/or mechanical stabilization.

The device 2 can be implanted in a bone, such as the femur 202a, as shown. The device 2 can be implanted closer to the hip joint 204 or, for example, in any location where delivery of a bioactive agent is desired. The device 2 can be coated with the agent. The device 2 can be loaded with one or more additional bioactive agents.

FIGS. 79a and 79b illustrate that the delivery device 2 can be used to fixably or removably anchor tendon to bone, such as into the humerus 202b and the ulna and/or radius 202c. One or more expandable delivery devices 2 can be inserted into a tendon 206. The delivery device 2 can be a radially expanding or unexpanding anchor. The delivery device 2 can be a tether. The device 2 can be located entirely within a tendon and/or bone adjacent to the tendon and/or other surrounding tissue. The delivery device 2 can be initially positioned in the tendon and/or bone in a radially contracted configuration. The delivery device 2 can then be radially expanded, for example, fixing the tendon to the bone. The radial expansion of the delivery device 2 can expand the size of the longitudinal channel 8. Before or after positioning and/or radially expanding the delivery device 2, the longitudinal channel 8 can be left empty or filled with one or more agents, fillers, or any other material disclosed herein (e.g., BMP, bone chips, morselized bone, autograft, allograft, xenograft, combinations thereof). The longitudinal channel 8 can be in fluid communication with the surrounding tissue, such as the soft tissue (e.g., ligaments and/or tendons) and/or bones and/or body fluids (e.g., blood, synovial fluid). A deployment tool 210 can deliver agents, fillers or any other materials disclosed herein to the target site, such as in the longitudinal channel 8 and/or elsewhere in and/or around the delivery device 2.

The delivered agents, fillers, or any other materials disclosed herein can be either pre-loaded on or in the delivery device 2 or placed into the longitudinal channel after the delivery device has been radially expanded in vivo. The delivery device 2 can be a hollow screw or anchor (e.g., expandable or non-expandable). The agents, fillers, or any, other materials disclosed herein can elute or otherwise flow from the delivery device 2, for example through the ingrowth ports 14, to the surrounding tissue (e.g., tendon, ligament, bone, cartilage, tendon, body fluids, combinations thereof).

FIG. 80 shows a delivery device 2 deployed at an anterior cruciate ligament (ACL) 208. The delivery device 2 can be deployed between two torn sections of the ACL 208. A first end of the delivery device 2 can be anchored to a first section of a damaged ACL. A second end of the delivery device 2 can be anchored to a second section of a damaged ACL. For example, the frayed terminal ends of the damaged ACL sections can be packed within the longitudinal channel 8 or otherwise in the radial interior of the delivery device 2. For example, the delivery device 2 can then be radially contracted (e.g., securely compressing and gripping the ACL in the longitudinal channel 8).

Also for example, the terminal ends of the damaged ACL sections can be attached to the exterior of the radial exterior of the delivery device 2, as shown. The delivery device 2 can fix the first section of the damaged ACL to the second section of the damaged ACL. The delivery device 2 can be located entirely within the damaged ACL 208 and/or located around an ACL graft (e.g., a patellar tendon autograft, allograft or xenograft).

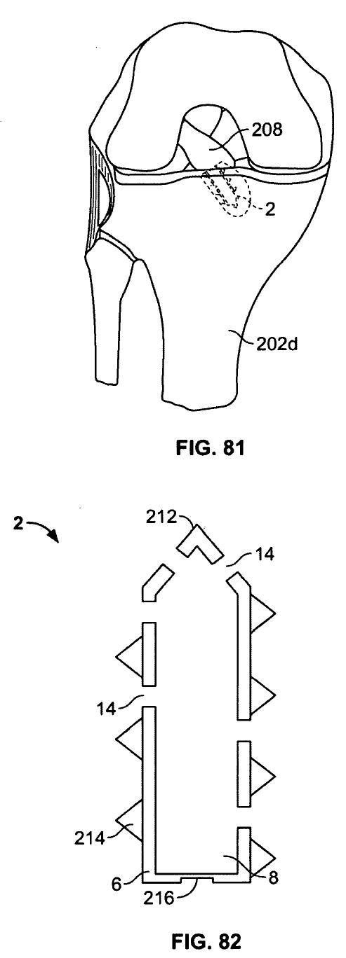

FIGS. 81 and 82 illustrate that the delivery device can have a sharpened tip The expandable support device can have one or more transverse or helical threads 214. The threads 214 can be configured to facilitate screwing the delivery device 2 into a target site. The delivery device 2 can have a screwdriver or other tool port 216. The tool port 216 can be configured to receive a rotation and/or translation tool (e.g., screwdriver). As shown in FIG. 81, the delivery device 2 can be used to anchor an ACL 208 in the tibia 202d (and any other ligament in any other bone). The delivery device 2 can be radially expanded after or during screwing or otherwise positioning the deliver) device adjacent to the ACL 208 in the tibia 202d.

The expandable delivery device 2 can be placed in the vertebral bodies, bones of the hand and/or finger, long bones, or combinations thereof.

The expandable delivery devices 2 can be deployed into an existing bone tunnel or into a tunnel formed by a drill, tamp, reamer (e.g., to remove more bone), or combinations thereof. The expandable delivery devices 2 can act as a tool to position the expandable delivery devices 2 within the fracture, for example, and then expand the distal end of the expandable delivery devices 2 to stabilize. The expandable delivery devices 2 can be threaded into place (e.g., self-deployed without a pre-formed tunnel or with a completely or partially pre-formed tunnel). One or two ends of the device 2 can be threaded. The threads can be on the radial interior and/or exterior of the delivery device 2. Multiple threads can be oriented in the same or different directions (e.g., to prevent backing-out of tissues on opposite sides of the delivery device). The expandable delivery devices 2 can be expanded at either end first (e.g., to align a fracture plane), in the center first, at both ends concurrently, or concurrently along the entire length. The expandable delivery devices 2 can self-anchor. The expandable delivery devices 2 can be anchored to surrounding tissue with a separate device (e.g., peg, brad, hook, thread, or combinations thereof.

The expandable delivery devices 2 can be filled, for example in the longitudinal channel 8 and/or in the ingrowth ports 14, with bone chips, cement, drugs, polymers, other metal structures, mixes of all theses and/or bioactive agents as described herein. The expandable delivery devices 2 can be filled before or after the expandable delivery device 2 is radially expanded at the target site, and/or before the expandable delivery device 2 is positioned at the target site. Any of the materials on or on the delivery device 2 can elute, leech, flow or otherwise exit the device 2 through the ingrowth ports 14, the longitudinal channel 8, or via micropores in the wall 6, out of a coating (e.g., a polymer or cloth, or any other coating described herein) on the surface of the delivery device 2, or combinations thereof. The expandable delivery devices 2 can be radiopaque. The expandable delivery devices 2 can provide a stabilizing force to the surrounding tissue.

The expandable delivery devices 2 can be covered with a polymer and/or a vessel or chamber to hold one or more agents (e.g., drugs). The expandable delivery devices 2 can be removed from the target site (e.g., bone), for example, by radially contracting the expandable support device 2. The expandable delivery device 2 can be radially contracted and repositioned at the target site, for example, if placement or sizing errors occur. The expandable delivery device 2 can be removed from the target site after a desired healing takes place.

Any or all elements of the expandable delivery devices 2, supports, or stents and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON®. from E. I. Du Pont de Nemours and Company, Wilmington, Del.), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France). aliphatic polyether polyurethanes (e.g., TECOFLEX® from Thermedics Polymer Products, Wilmington, Mass.), polyvinyl chloride (PVC), polyurethane, thermoplastic, fluorinated ethylene, propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the expandable delivery devices 2, supports, or stents and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

Any of the expandable delivery devices 2, supports, or stents and/or elements of the expandable delivery devices 2, supports, or stents could be made from a biodegrading polymer as well. In such a case, the bioactive agents could be in the polymer, on the polymer, or on the bore of the vehicle. The bioactive agents and/or carrier would be designed to slowly elute from the vehicle.

The expandable delivery devices 2, supports, or stents and/or elements of the expandable delivery devices, supports, or stents and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E2 Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

We claim:

1. A method for delivering an agent to an orthopedic target site located in biological tissue, the method comprising:
positioning a radially expandable delivery device comprising the agent at the target site; and
positioning the agent at the target site;
wherein the target site is selected from a group consisting of a bone, a cartilage, a tendon, and a ligament.

2. The method of claim 1, wherein the positioning comprises clearing at least some of the tissue from a volume within the target site using the expandable delivery device.

3. The method of claim 1, further comprising physically stabilizing the target site with the expandable delivery device.

4. The method of claim 1, further comprising radially expanding the expandable delivery device.

5. The method of claim 4, wherein the radially expanding further comprises clearing at least some of the tissue from a volume within the target site.

6. The method of claim 1, where the target site comprises the femur.

7. The method of claim 1, where the target site comprises a vertebral body.

8. The method of claim 1, where the target site comprises an anterior cruciate ligament.

9. The method of claim 1, where the target site comprises a bone in a hand.

10. The method of claim 1, where the expandable delivery device comprises a cavity comprising the agent.

11. The method of claim 1, where the expandable delivery device comprises a matrix comprising the agent.

12. The method of claim 1, where the expandable delivery device is coated with a polymer containing the agent.

13. The method of claim 1, further comprising radially contracting the expandable delivery device.

14. The method of claim 13, further comprising repositioning the expandable delivery device and radially expanding the expandable delivery device.

15. An expandable delivery device for delivering an agent to a biological target site comprising:
a first strut;
a wall, wherein the wall defines a central channel and wherein the wall has ports therethrough; and
a filler;
wherein the central channel is substantially completely filled by the filler.

16. The device of claim 15, further comprising a helical thread.

17. The device of claim 15, further comprising a sharpened distal tip.

18. The device of claim 15, further comprising a matrix, wherein the filler is in the matrix.

19. The device of claim 18, further comprising a polymer coating, wherein the matrix is in the polymer coating.

20. The device of claim 15, wherein the filler comprises bone protein.
